(19) United States
(12) Patent Application Publication
(10) Pub. No.: US 2014/0104569 A1
(43) Pub. Date: Apr. 17, 2014

(54) OPHTHALMIC APPARATUS AND WAVEFRONT ABERRATION CORRECTION METHOD THEREFOR

(71) Applicant: Canon Kabushiki Kaisha, Tokyo (JP)

(72) Inventor: Shoichi Yamazaki, Yokohama-shi (JP)

(73) Assignee: Canon Kabushiki Kaisha, Tokyo (JP)

(21) Appl. No.: 14/048,143

(22) Filed: Oct. 8, 2013

(30) Foreign Application Priority Data
Oct. 17, 2012 (JP) 2012-230262

(51) Int. Cl. A61B 3/14

(52) U.S. Cl. CPC A61B 3/14
USPC 351/206; 351/246

(57) ABSTRACT

An ophthalmic apparatus which captures an image of a retina by illuminating the fundus of an eye to be examined with light from a light source through a measuring optical path is provided. The ophthalmic apparatus divides light from the light source into light propagating to the measuring optical path and light propagating to a reference optical path different from the measuring optical path, obtains interference light by composing return light returned along the reference optical path and return light from the measuring optical path, images interference fringes generated by the interference light composed at a position conjugate with the position of the pupil of the eye, and corrects a wavefront aberration of light passing through the measuring optical path by using correction data for correcting a wavefront aberration of the eye which is obtained from a captured image of interference fringes.

### BACKGROUND OF THE INVENTION

1. Field of the Invention

The present invention relates to an ophthalmic apparatus which measures the wavefront aberration generated in the eyeball of an individual and forms an image upon cancellation of the aberration and a wavefront aberration correction method therefor.

2. Description of the Related Art

Conventionally, there are available ophthalmic apparatuses which image the fundus by using OCT (Optical Coherence Tomography) which acquires a fundus tomographic image by using optical coherence or SLO (Scanning Laser Ophthalmoscope) which acquires a high-precision two-dimensional image of the fundus. These ophthalmic apparatuses need to capture images through optical tissues of the eyes such as the corneas and crystal lenses.

Recently, with increases in image resolution, the aberrations of the corneas and crystal lenses greatly influence the image quality of captured images. Under the circumstances, studies have been made on AO-OCT and AO-SLO in which each optical system incorporates an AO (Adaptive Optics) as a compensation optical system which measures the aberration of the eye and corrects the aberration.

The technique disclosed in Japanese Patent Laid-Open No. 2010-279681 uses both the AO-OCT and AO-SLO described above. This patent literature also discloses a Shack-Hartmann wavefront sensor which is often generally used as an eye aberration measuring device. This technique obtains excellent OCT and SLO images by performing focusing so as to cancel the eyeball aberration measured by the Shack-Hartmann wavefront sensor or correcting the eyeball aberration by using a deformable mirror. According to the description of the patent literature, a liquid crystal element may be used instead of the deformable mirror.

In general, a Shack-Hartmann wavefront sensor includes an imaging device such as a CCD or CMOS and a two-dimensional microlens array having a plurality of microlenses arrayed. The imaging device is placed at the focal plane of the microlenses. The microlens array includes about several thousand microlenses, with about several ten microlenses being arrayed in the vertical direction and about several ten microlenses being arrayed in the horizontal direction. The imaging device (having 1,000,000 pixels or more; there is also currently available an imaging device having 10,000,000 pixels or more) is placed below the microlenses, with an imaging device area of 10×10 pixels or more being assigned below one microlens.

Note that since an ocular pupil plane and the microlens planes of the Shack-Hartmann wavefront sensor are set to be almost conjugate with each other, the wavefront aberration of the ocular pupil plane directly appears on the microlens planes. Note, however, that since the microlens array is constituted by several 10 microlenses in the vertical direction and several ten microlenses in the horizontal direction, the sensor measures the wavefront aberration divided in the vertical and horizontal directions.

On one microlens, a focus image shifts vertically and horizontally on the assigned imaging device area depending on the state of wavefront aberration. One microlens can therefore measure the aberration amount of the eyeball based on the shift amounts. Thereafter, the eyeball aberrations measured by all the microlenses (several thousands) are combined by approximation (formed into Zernike coefficients) to calculate the aberration of the entire eyeball surface. This is the principle of a Shack-Hartmann wavefront sensor.

Most of the current deformable mirrors functioning as wavefront correction devices are driven while being divided into several ten parts in the vertical direction and several ten parts in the horizontal direction. Such a mirror can perform wavefront correction by several thousand division driving but it is difficult to perform driving with more divisions.

Liquid crystal elements used in place of deformable mirrors includes a special phase type liquid crystal element capable of controlling phases, which can control the phase of each pixel. “Capable of controlling phases” herein indicates that each pixel can perform wavefront correction.

An existing phase type liquid crystal element is a device having 1,000 pixels in the vertical direction and 1,000 pixels in the horizontal direction, and can perform wavefront correction driving with a total of 1,000,000 pixels. Note that since the wavefront correction device is also placed at a position conjugate with the ocular pupil plane, this operation is equivalent to wavefront aberration correction with the entire eyeball being divided into 1,000,000 parts.

As described above, using a phase type liquid crystal element as a wavefront correction device can perform wavefront correction on 1,000,000 pixels (=1,000,000 division wavefront correction). Even if a high-resolution phase type liquid crystal element is used as the above wavefront correction device, since a Shack-Hartmann wavefront sensor is limited to several thousand divisions, the number of divisions is not sufficient for the phase type liquid crystal element capable of 1,000,000 division driving. Furthermore, a high-division, high-resolution wavefront sensor is required. That is, the Shack-Hartmann wavefront sensor itself requires a high-resolution wavefront sensor capable of driving with 1,000,000 or more divisions.

In addition, the Shack-Hartmann wavefront sensor performs approximate fitting to Zernike aberration coefficients as a whole from divided wavefront aberration data, and inputs the resultant data to the wavefront correction device. However, approximate fitting causes errors and requires much computation processing time.

### SUMMARY OF THE INVENTION

According to one embodiment of the present invention there is provided an ophthalmic apparatus which can correct the wavefront aberration in the eyeball with high precision and high resolution.

According to one aspect of the present invention, there is provided an ophthalmic apparatus which captures an image of a retina by causing a light source to illuminate a fundus of an eye to be examined with light through a measuring optical path and guiding return light to a light receiving sensor through the measuring optical path, the apparatus comprising: a dividing unit configured to divide light from the light source into light propagating to the measuring optical path and light propagating to a reference optical path different from the measuring optical path and obtain interference light by composing return light returned along the reference optical path and return light from the measuring optical path; an imaging unit configured to image interference fringes generated by the interference light at a position conjugate with a position of a pupil of the eye; and a correction unit configured to correct a wavefront aberration of light passing through the measuring optical path by using correction data for correcting a wavefront aberration of the eye which is obtained from an image of interference fringes imaged by the imaging unit.

Furthermore, according to another aspect of the present invention, there is provided a wavefront aberration correction method for an ophthalmic apparatus which captures an image of a retina by causing a light source to illuminate a fundus of an eye to be examined with light through a measuring optical path and guiding return light to a light receiving sensor through the measuring optical path, the method comprising: dividing light from the light source into light propagating to the measuring optical path and light propagating to a reference optical path different from the measuring optical path and obtaining interference light by composing return light returned along the reference optical path and return light from the measuring optical path; imaging interference fringes generated by the interference light at a position conjugate with a position of a pupil of the eye; and correcting a wavefront aberration of light passing through the measuring optical path by using correction data for correcting a wavefront aberration of the eye which is obtained from a captured image of interference fringes.

Further features of the present invention will become apparent from the following description of exemplary embodiments with reference to the attached drawings.

### BRIEF DESCRIPTION OF THE DRAWINGS

FIG. 1 is a view showing an example of the arrangement of a wavefront aberration correction apparatus according to the first embodiment;

FIG. 2 is a flowchart showing wavefront aberration correction processing in this embodiment;

FIG. 3 is a view showing an example of the arrangement of a wavefront aberration correction apparatus according to a modification of the first embodiment;

FIG. 4 is a view showing an example of the arrangement of a wavefront aberration correction apparatus according to the second embodiment;

FIG. 5 is a view showing an example of the arrangement of a wavefront aberration correction apparatus according to a modification of the second embodiment;

FIG. 6 is a view showing an example of the arrangement of a wavefront aberration correction apparatus according to the third embodiment;

FIG. 7 is a view showing an example of the arrangement of a wavefront aberration correction apparatus according to a modification of the third embodiment;

FIGS. 8A and 8B are views showing an example of an interference map according to this embodiment;

FIG. 9A is a view showing an example of wavefront aberration map according to this embodiment; and

FIG. 9B is a view showing a wavefront aberration cancel map according to the embodiment.

### DESCRIPTION OF THE EMBODIMENTS

Modes for carrying out the present invention will be described below in detail below with reference to the accompanying drawings. The following embodiments each will exemplify a fundus tomography apparatus using an OCT (Optical Coherence Tomography) as an ophthalmic apparatus. This apparatus accurately measures wavefront aberrations by using an interference fringe imaging camera (interference fringe sensor) for measuring wavefront aberrations. The resolution of wavefront aberration measurement is equal to or more than the resolution (1,000,000 pixels) of wavefront correction device for correcting wavefront aberrations.

This embodiment will exemplify an AO-OCT incorporating, in an optical system, an AO (Adaptive Optics) as a compensation optical system for correcting the wavefront aberration of the eyeball of the eye to be examined.

### First Embodiment

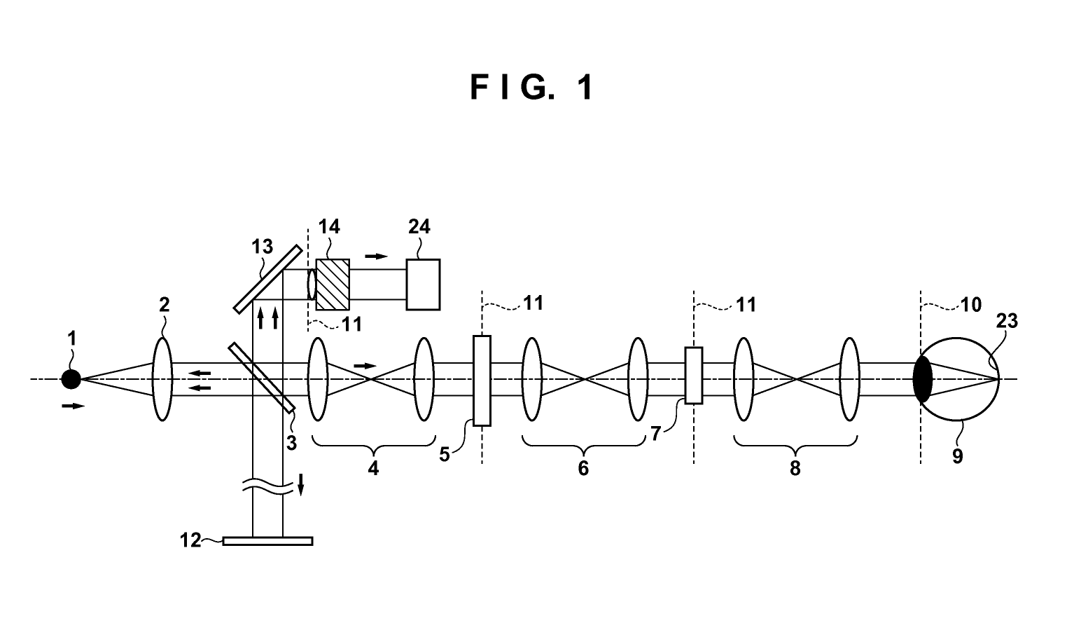

The arrangement of a wavefront aberration correction apparatus according to the first embodiment will be described with reference to FIG. 1. The light emitted by a light source (not shown) and propagating through an optical fiber emerges from a flat end face (FC) or tilt end face (APC) of an optical fiber, serving as a light-emitting surface. On the other hand, return light from the eyeball enters the optical fiber with the flat end face (FC) or tilt end face (APC) of the optical fiber, serving as a light-receiving surface. The return light which has entered the optical fiber is guided to a light receiving sensor such as a PD (PhotoDiode) through a fiber coupler (not shown), and an output from the light receiving sensor is formed into a retina image. Referring to FIG. 1, this arrangement forms three conjugate planes 11 conjugate with an eyeball pupil 10 through three afocal conjugate optical systems 4, 6, and 8. An interference fringe sensor 14, a wavefront correction device 5, and a scanner 7 are respectively arranged at the three conjugate planes.

The light coming from the light source and reaching an end face 1 of the optical fiber is collimated by a collimator lens 2 and enters a light dividing element (half mirror) 3. The light transmitted through the light dividing element 3 is guided to a measuring optical path (forward path). The transmitted light enters the wavefront correction device 5 through the afocal conjugate optical system 4. Although FIG. 1 exemplifies a transmission type wavefront correction device 5, a reflection and phase phase type liquid crystal element capable of performing correction with high resolution may be used. The light emerging from the wavefront correction device 5 enters the scanner 7 through the afocal conjugate optical system 6. The scanner 7 scans the fundus in the eyeball (retina 23 in FIG. 1) with incident light.

Although FIG. 1 exemplifies the transmission type scanner 7, a reflection type scanning mirror which is two-dimensionally driven or the like may be used. Note that a scanning mirror to be two-dimensionally driven may be implemented by combining two one-dimensional scanning mirrors. FIG. 1 shows a central light beam as a scanning range with respect to the retina 23.

The transmitted light then illuminates an eyeball 9 through the afocal conjugate optical system 8 and the eyeball pupil 10. This light is reflected by the retina 23 to become return light. The return light propagates backward along the measuring optical path (forward path) as a backward path.

Of the light collimated by the collimator lens 2, the light reflected by the light dividing element 3 is guided as reference light to a reference optical path (forward path). The reference light propagates to a return reference mirror 12 along the reference optical path. This reflected light returns as return light in the opposite direction along the reference optical path as a backward path. An optical path adjusting unit (not shown) in the reference optical path adjusts the optical path length from the light dividing element 3 to the return reference mirror 12 so as to make the length become almost equal to the optical path length from the light dividing element 3 in a measuring optical path to the retina 23. This can increase the coherency of interference fringes between return light along the measuring optical path and return light along the reference optical path which are divided by the light dividing element 3.

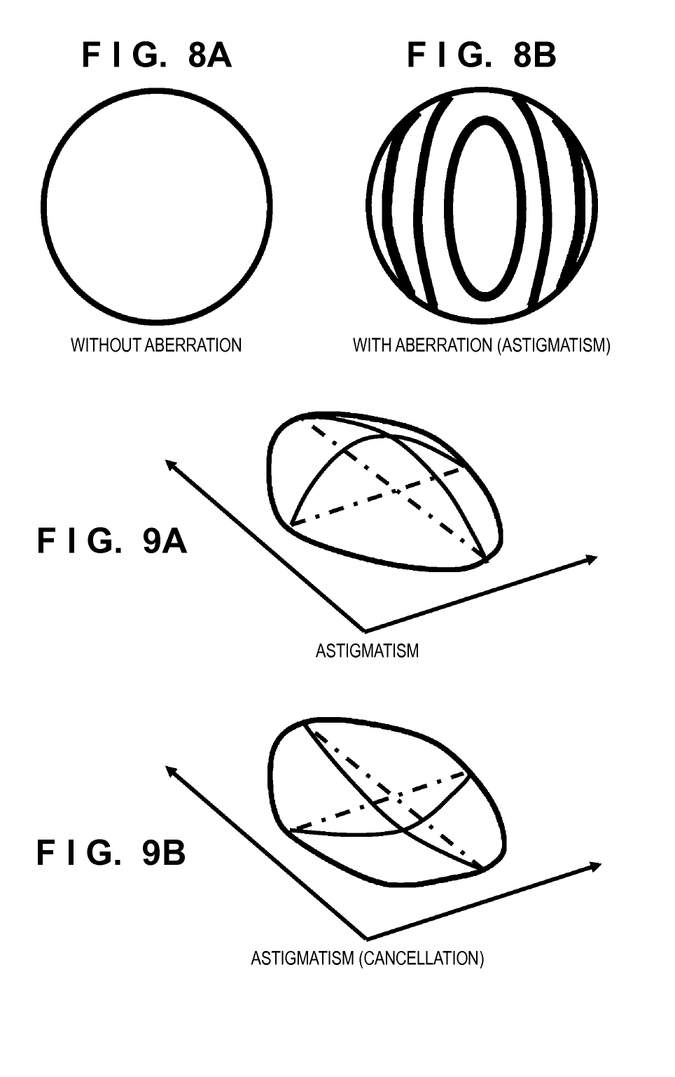

The light dividing element 3 generates interference light by composing return light, of the return light along the reference optical path, which is transmitted through the light dividing element 3 with return light, of the return light along the measuring optical path, which is reflected by the light dividing element 3. In this case, the interference light enters the interference fringe sensor 14 though a mirror 13. When the optical path length of the reference optical path coincides with that of the measuring optical path, the interference fringe sensor 14 can capture an interference fringe image having high contrast with the interference light. This improves the precision of wavefront aberration measurement. FIGS. 8A and 8B show an example of an interference fringe image captured by the interference fringe sensor 14. If the eyeball has no aberration, return light along the measuring optical path and return light along the reference optical path generate almost no interference fringes (FIG. 8A). If the eyeball has aberration, interference fringes are generated (FIG. 8B).

The interference fringe image captured by the interference fringe sensor 14 is sent to a wavefront aberration calculation unit 24 to form the image into a contour map (wavefront aberration map) and an opposite phase map (wavefront aberration cancel map). The resultant data is directly sent to the wavefront correction device 5 to correct wavefront aberrations. This makes it possible to correct wavefront aberrations at a high processing speed by simple conversion computation without including errors.

The interference light generated by the light of return light along the reference optical path which is reflected by the light dividing element 3 and the light of return light along the measuring optical path which is transmitted through the light dividing element 3 enters the end face 1 through the collimator lens 2. This light is then guided to the light receiving sensor. The interference light signal detected by the light receiving sensor and output is used to form a tomographic image. When using an SS (SWEPT SOURCE) light source designed to generate light which has high coherence and changes in wavelength with time, the apparatus obtains a retina tomographic image by performing Fourier transform of the data (interference light signal) obtained by a PD (PhotoDiode) as a light receiving sensor. If the light source to be used is a low-coherence SLD, the apparatus obtains a retina tomographic image by performing spectroscopy before a line CCD sensor as a light receiving sensor and performing Fourier transform of data from the line CCD sensor.

Using the light dividing element (half mirror) 3 in the above manner allows to use the measuring optical path and reference optical path for OCT fundus tomographic images, which the ophthalmic apparatus originally has, as a measuring optical path and a reference optical path for the interference fringe sensor 14. This makes it possible to increase the resolution of the wavefront sensor without increasing the size of the apparatus. In addition, it is possible to calculate and update correction data for the wavefront correction device 5 from the interference fringe image obtained during OCT imaging and to effectively remove the influence of wavefront aberrations.

Note that an SS light source is preferably selected as a light source to be used. Using light with various wavelength components like SLO light (light with wavelength width of about 100 nm centered on single reference wavelength=wavelength with highest intensity) will decrease the reliability of measurement of wavefront aberrations because the interference fringe image on the interference fringe sensor 14 has poor image quality. In contrast to this, an SS light source can emit light with almost single wavelength while changing the wavelength with time instead of emitting light with a large wavelength width. This makes it possible to obtain excellent interference fringes with high coherence. It is possible to perform proper interference fringe measurement with a single reference wavelength of 1,030 nm exhibiting a highest intensity by using an SS light source with, for example, a reference wavelength of 1,030 nm and a variable wavelength width of 70 nm. In addition, it is possible to also correct the chromatic aberration of the eyeball by acquiring interference fringe data a plurality of number of times with a plurality of single wavelengths different from the single reference wavelength and performing aberration correction for each wavelength by time-divisional wavelength sweeping of the SS light source upon wavefront measurement with each wavelength.

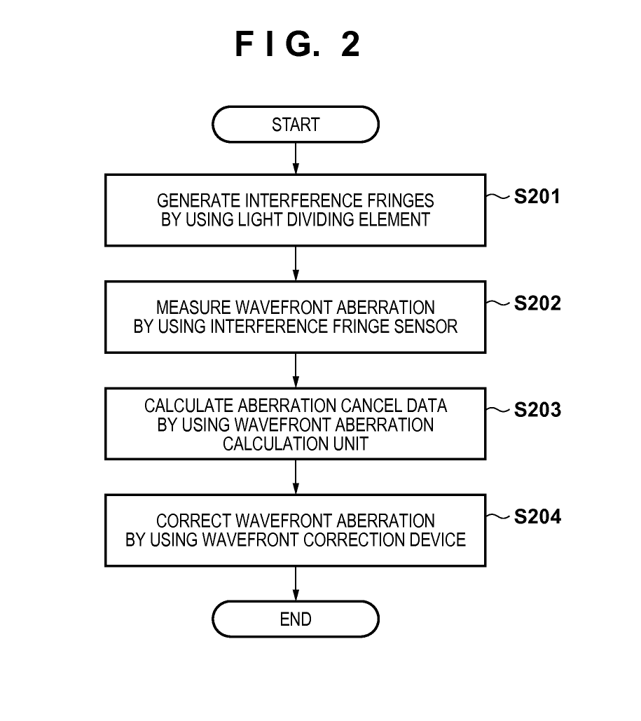

Wavefront aberration correction processing performed by the wavefront correction device 5 to correct the wavefront aberration in the eyeball which is measured by using the light dividing element 3 and the interference fringe sensor 14 will be described next with reference to the flowchart shown in FIG. 2.

First of all, in step S201, the light dividing element 3 divides light from the light source into a measuring optical path and a reference optical path, and the light dividing element 3 composes return light from the retina 23 and return light from the return reference mirror 12 to generate interference fringes. In step S202, the interference fringe sensor 14 images the interference fringes generated in step S201 to measure the wavefront aberration in the eyeball as a high-precision interference fringe image. In step S203, the wavefront aberration calculation unit 24 calculates wavefront aberration cancel data for canceling the wavefront aberration from the interference fringe image measured by the interference fringe sensor 14. In step S204, the wavefront correction device 5 corrects the wavefront aberration by using the wavefront aberration cancel data sent from the wavefront aberration calculation unit 24.

The fringe intervals of the interference fringe map (FIG. 8B) obtained from the interference fringes imaged in step S202 correspond to a wavelength λ/2, and the intervals between the first and second fringes and between the first and third fringes respectively correspond to 3λ/2 and 5λ/2. Converting this interference fringe map into a contour map yields a wavefront aberration map (FIG. 9A). It is therefore possible to easily generate a wavefront aberration map from the interference fringes imaged by the interference fringe sensor 14. The resolution and precision of the map are determined by the number of pixels of the interference fringe sensor 14 (imaging device) which has imaged the interference fringes. Existing imaging devices have pixel counts of 1,000,000 or more, and hence it is possible to increase the wavefront aberration measurement precision as compared with the Shack-Hartmann wavefront sensor whose measurement precision is determined by the number of microlenses (the number of divisions).

If, for example, the imaging device of the interference fringe imaging camera (interference fringe sensor 14) has 1,000×1,000 pixels (1,000,000 pixels), wavefront aberration data (FIG. 9A) is obtained for each pixel, and a wavefront aberration cancel map (FIG. 9B) is obtained by generating data with a phase opposite to that of the data. Inputting this wavefront aberration cancel map as two-dimensional data for driving each pixel of a wavefront correction device liquid crystal (1,000×1,000 pixels) realizes high-precision wavefront correction with a resolution of one pixel. If, therefore, the number of pixels of the interference fringe sensor 14 is close to that the wavefront correction device, it is possible to control the wavefront correction device almost for each pixel by using the interference fringe image obtained by the interference fringe sensor 14 and to realize high-precision wavefront aberration correction.

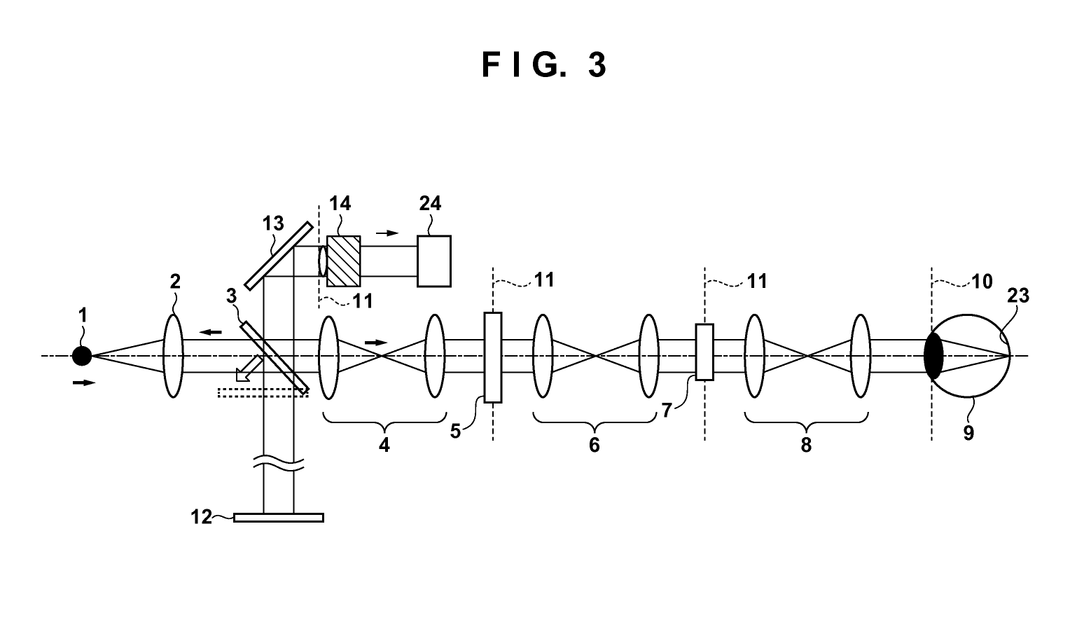

Although an AO-OCT has been exemplified as an ophthalmic apparatus, the present invention can be applied to an AO-SLO incorporating adaptive optics in the optical system of an SLO (Scanning Laser Ophtalmoscope) which captures a high-precision two-dimensional image of the fundus. FIG. 3 shows such an application example. The basic arrangement of the AO-SLO is the same as that of the AO-OCT in FIG. 1.

When using the AO-SLO, since reference light entering the end face 1 which generates an SLO image degrades the image, the light dividing element 3 is moved to a position where the element is not inserted in the optical path as indicated by the dotted line in FIG. 3. This arrangement makes it possible to obtain a proper SLO image without entrance of reference light. Note that when measuring the wavefront aberration in the eyeball, the light dividing element 3 is inserted in the optical path, as described with reference to FIG. 1. In addition, a control unit (not shown) may perform control to determine whether to use the light dividing element 3, at either the timing of the start of wavefront aberration measurement or the timing of the start of imaging of a fundus tomographic image.

Both the AO-OCT and the AO-SLO can capture a high-precision fundus tomographic image by measuring the wavefront aberration in the eyeball using the light dividing element 3 and the interference fringe sensor 14 and correcting the wavefront aberration using the wavefront correction device 5.

### Second Embodiment

The second embodiment of the present invention will be described in detail next with reference to the accompanying drawings. In the second embodiment, a light source for the acquisition of a fundus image for an AO-OCT has a wavelength different from that of a light source (beacon light source) for wavefront measurement for an interference fringe sensor 14.

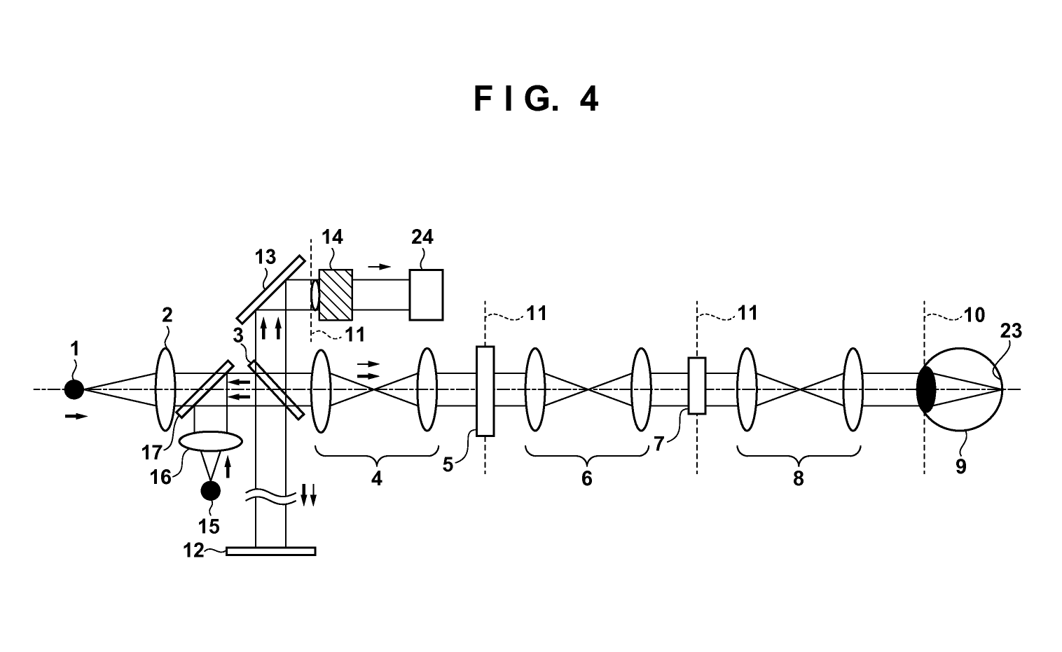

The arrangement of a wavefront aberration correction apparatus according to the second embodiment will be described with reference to FIG. 4. In this case, a beacon light source 15, a beacon collimator lens 16 which collimates light from the beacon light source 15, and a dichroic mirror 17 which reflects light with a beacon wavelength and transmits light with an OCT image wavelength are interposed between a collimator lens 2 and a light dividing element (half mirror) 3. This arrangement makes it possible to cut beacon ghost light by inhibiting beacon light from entering an end face 1 as a light receiving surface, thereby obtaining a high-quality fundus tomographic image. Note that wavefront aberration correcting operation is the same as that in the first embodiment except that beacon light is used for imaging interference fringes using the interference fringe sensor 14. Note that it is possible to turn off the beacon light source 15 during OCT imaging.

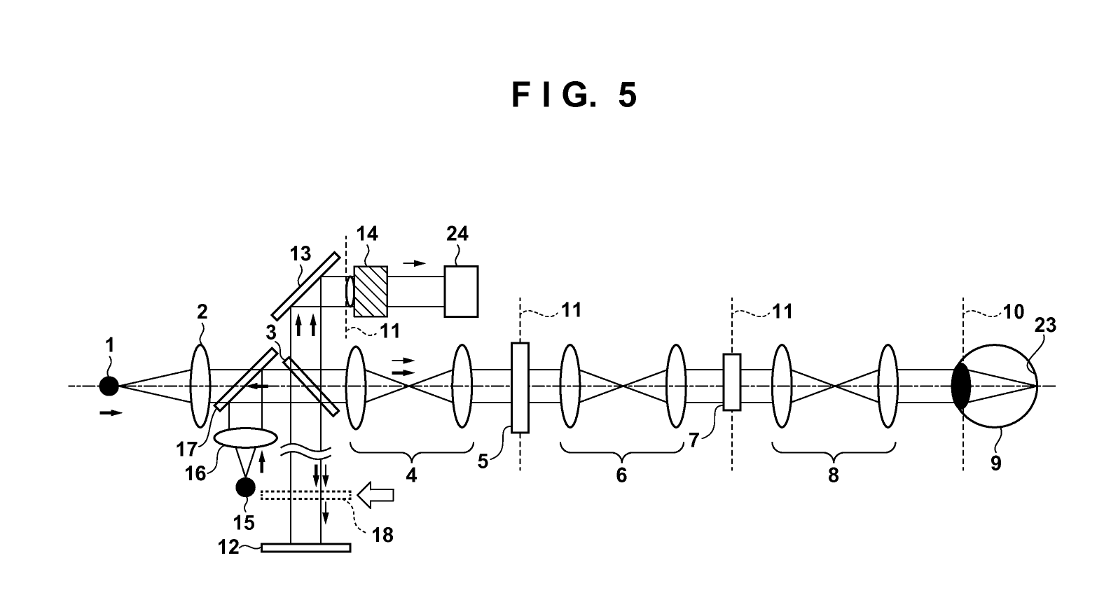

As in the first embodiment, the arrangement using the beacon light source 15 can also be applied to the AO-SLO. FIG. 5 shows an example of this arrangement. In the case shown in FIG. 5, a band-pass filter 18 which transmits light with a beacon wavelength and cuts off light with an SLO image wavelength is inserted in a reference optical path so as to inhibit reference light from a return reference mirror 12 from entering the end face 1 of the optical fiber. This arrangement makes it possible to prevent the generation of a ghost by inhibiting reference light from entering the end face 1 of the optical fiber, thereby obtaining an excellent SLO image. Note that it is possible to retract the dichroic mirror 17 from the optical path, instead of inserting the band-pass filter 18, to inhibit light from the beacon light source 15 from entering the measuring optical path, and to retract the light dividing element 3 from the optical path to disconnect the reference optical path.

In addition, even if the band-pass filter 18 is inserted, since the reference optical path for beacon light exists, the interference fringe sensor 14 can capture an interference fringe image. This makes it possible to obtain a proper SLO image and perform high-resolution, high-precision wavefront aberration measurement and wavefront aberration correction.

### Third Embodiment

The third embodiment of the present invention will be described in detail next with reference to the accompanying drawings. In the second embodiment, the end face 1 of the optical fiber is used as a light emitting surface and a light receiving surface. In contrast, the third embodiment uses an end face of an optical fiber as a light emitting surface 19 and also uses a light receiving surface 22 separated from the light emitting surface.

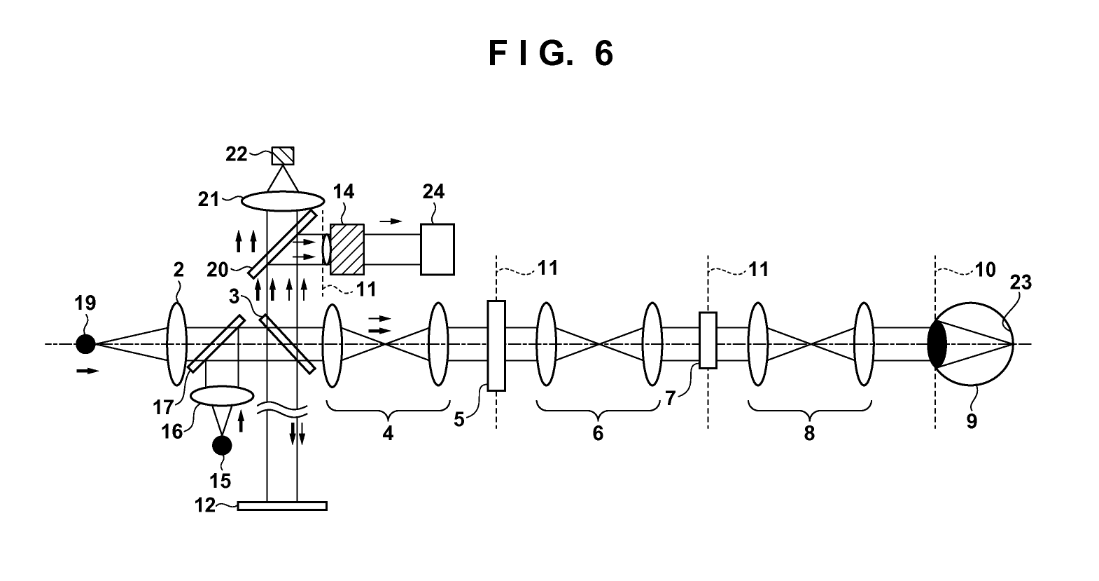

The arrangement of a wavefront aberration correction apparatus according to the third embodiment will be described with reference to FIG. 6. This arrangement differs from the arrangement shown in FIG. 4 in that an end face 1 is used as the light emitting surface 19, the light receiving surface 22 is separately provided, the mirror 13 is replaced by a dichroic mirror 20 which reflects light with a beacon wavelength and transmits light with an OCT image wavelength, and a light receiving lens 21 is inserted between the dichroic mirror 20 and the light receiving surface 22. In the arrangement of the second embodiment, a ghost (return light reflected by the surface of the collimator lens 2 from the light emitting surface) tends to be generated between the end face 1 serving as the light emitting surface and the light receiving surface and the collimator lens 2, leading to a deterioration in an OCT image. However, separating the light emitting surface 19 from the light receiving surface 22 as in the third embodiment can prevent such a ghost from entering the light receiving surface, thereby obtaining an OCT image with higher image quality.

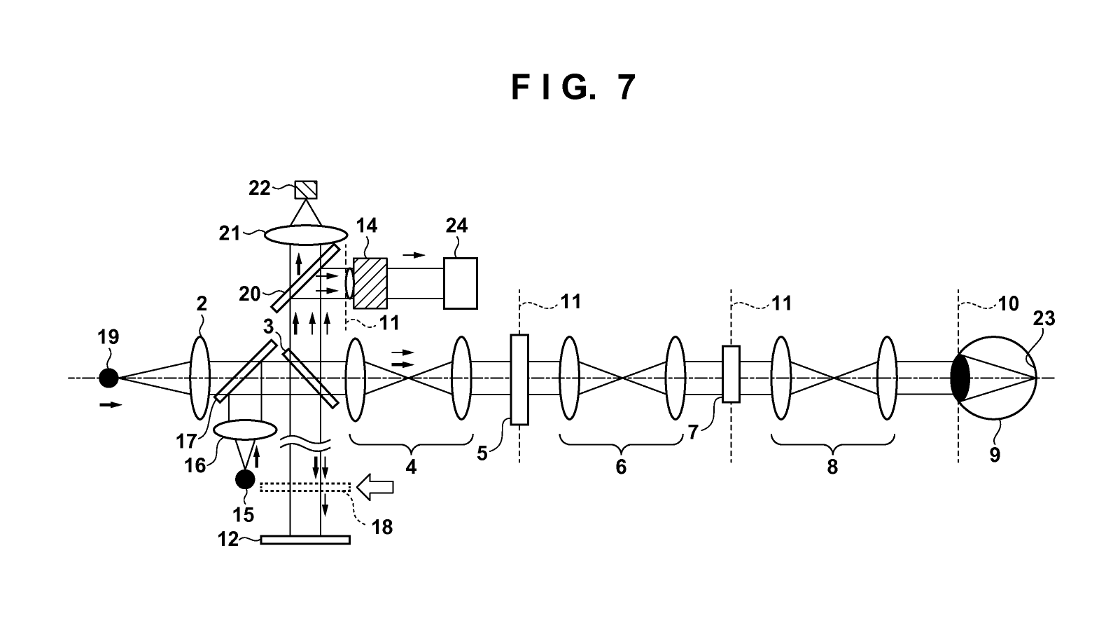

Like the second embodiment, the above arrangement can also be applied to an AO-SLO. In the case shown in FIG. 7, as in the second embodiment shown in FIG. 5, a band-pass filter 18 which transmits light with a beacon wavelength and cuts off light with an SLO image wavelength is inserted in a reference optical path. Note that the effects of the third embodiment are the same as those of the second embodiment. As is obvious, the arrangement described in the third embodiment which is configured to separate the light emitting surface 19 from the light receiving surface 22 can be applied to the arrangement of the first embodiment (FIGS. 1 and 3). In addition, as is obvious, the dichroic mirror 20 may be configured to reflect light with an OCT image wavelength and transmit light with a beacon wavelength.

As has been described above, according to the first to third embodiments, it is possible to correct the wavefront aberration in the eyeball with high precision and high resolution.

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-230262, filed Oct. 17, 2012, which is hereby incorporated by reference herein in its entirety.

What is claimed is:

1. An ophthalmic apparatus which captures an image of a retina by causing a light source to illuminate a fundus of an eye to be examined with light through a measuring optical path and guiding return light to a light receiving sensor through the measuring optical path, the apparatus comprising:
a dividing unit configured to divide light from the light source into light propagating to the measuring optical path and light propagating to a reference optical path different from the measuring optical path and obtain interference light by composing return light returned along the reference optical path and return light from the measuring optical path;
an imaging unit configured to image interference fringes generated by the interference light at a position conjugate with a position of a pupil of the eye; and
a correction unit configured to correct a wavefront aberration of light passing through the measuring optical path by using correction data for correcting a wavefront aberration of the eye which is obtained from an image of interference fringes imaged by said imaging unit.

2. The apparatus according to claim 1, wherein said light dividing unit guides the interference light to an optical path extending to said imaging unit.

3. The apparatus according to claim 1, wherein said light dividing unit guides the interference light to an optical path extending to said imaging unit and to an optical path extending to the light receiving sensor.

4. The apparatus according to claim 1, wherein said light dividing unit moves to a position where said light dividing unit is not inserted in the measuring optical path, when an image of the retina is captured by the light receiving sensor.

5. The apparatus according to claim 1, wherein the light source comprises a light source configured to output light upon switching between a plurality of wavelengths, and
said correction unit uses correction data obtained from an image of interference fringes imaged with light having one of a plurality of wavelengths output from the light source.

6. The apparatus according to claim 1, wherein the light source comprises a light source configured to output light upon switching between a plurality of wavelengths, and
said correction unit corrects a wavefront aberration for each wavelength by using correction data obtained from each of images of interference fringes obtained by using light of a plurality of wavelengths.

7. The apparatus according to claim 1, further comprising:
a light source different from the light source; and
a unit configured to guide light from the different light source to said light dividing unit through a dichroic mirror which transmits light from the light source and reflects light from the different light source,
wherein said imaging unit images interference fringes generated by return light of light from the different light source on the measuring optical path and the reference optical path.

8. The apparatus according to claim 7, further comprising a band-pass filter placed on the reference optical path and configured to cut off light from the light source and transmit light from the different light source.

9. The apparatus according to claim 7, further comprising another dichroic mirror placed on an optical path of interference light generated by said light dividing unit and configured to separate light from the light source from light from the different light source,
wherein interference light by light from the different light source which is separated by said another dichroic mirror is guided to said imaging unit, and light from the light source which is separated by said another dichroic mirror is guided to the light receiving sensor.

10. A wavefront aberration correction method for an ophthalmic apparatus which captures an image of a retina by causing a light source to illuminate a fundus of an eye to be examined with light through a measuring optical path and guiding return light to a light receiving sensor through the measuring optical path, the method comprising:
dividing light from the light source into light propagating to the measuring optical path and light propagating to a reference optical path different from the measuring optical path and obtaining interference light by composing return light returned along the reference optical path and return light from the measuring optical path;
imaging interference fringes generated by the interference light at a position conjugate with a position of a pupil of the eye; and
correcting a wavefront aberration of light passing through the measuring optical path by using correction data for correcting a wavefront aberration of the eye which is obtained from a captured image of interference fringes.

11. A non-transitory computer-readable storage medium storing a program for causing a computer to execute a wavefront aberration correction method defined in claim 10.
